SEQUENCE LISTING (7 sequences)

SEQ ID NO: 1 - protein, 614 aa, Homo sapiens
MRPPQCLLHTPSLASPLLLLLLWLLGGGVGAEGREDAELLVTVRGGRLRGIRLKTPGGPVSAFLGIPFAEPPMGPRRFLPPEPKQPWSGVVDATTFQSVCYQYVDTLYPGFEGTEMWNPNRELSEDCLYLNVWTPYPRPTSPTPVLVWIYGGGFYSGASSLDVYDGRFLVQAERTVLVSMNYRVGAFGFLALPGSREAPGNVGLLDQRLALQWVQENVAAFGGDPTSVTLFGESAGAASVGMHLLSPPSRGLFHRAVLQSGAPNGPWATVGMGEARRRATQLAHLVGCPPGGTGGNDTELVACLRTRPAQVLVNHEWHVLPQESVFRFSFVPVVDGDFLSDTPEALINAGDFHGLQVLVGVVKDEGSYFLVYGAPGFSKDNESLISRAEFLAGVRVGVPQVSDLAAEAVVLHYTDWLHPEDPARLREALSDVVGDHNVVCPVAQLAGRLAAQGARVYAYVFEHRASTLSWPLWMGVPHGYEIEFIFGIPLDPSRNYTAEEKIFAQRLMRYWANFARTGDPNEPRDPKAPQWPPYTAGAQQYVSLDLRPLEVRRGLRAQACAFWNRFLPKLLSATDTLDEAERQWKAEFHRWSSYMVHWKNQFDHYSKQDRCSDL

SEQ ID NO: 2 - protein, 30 aa, Homo sapiens
KAEFHRWSSYMVHWKNQFDHYSKQDRCSDL

SEQ ID NO: 3 - protein, 14 aa, Homo sapiens
AEFHRWSSYMVHWK

SEQ ID NO: 4 - protein, 15 aa, Homo sapiens
NQFDHYSKQDRCSDL

SEQ ID NO: 5 - protein, 7 aa, Homo sapiens
SYMVHWK

SEQ ID NO: 6 - protein, 4 aa, Homo sapiens
VHWK

SEQ ID NO: 7 - protein, 42 aa, Homo sapiens [sequence omitted]

(19) United States
(12) Patent Application Publication
(10) Pub. No.: US 2025/0277798 A1
(43) Pub. Date: Sep. 4, 2025

(54) SKIN BIOMARKER

(71) Applicant: NEURO-BIO LTD, Abingdon, Oxfordshire (GB)

(72) Inventors: Susan GREENFIELD, Abingdon, Oxfordshire (GB); Jason HARCUP, Trumbull, CT (US)

(21) Appl. No.: 18/696,950

(22) PCT Filed: Sep. 20, 2022
(86) PCT No.: PCT/GB2022/052364

§ 371 Date: Mar. 28, 2024

(30) Foreign Application Priority Data
Sep. 29, 2021 (GB) 2113941.5

(51) Int. Cl. G01N 33/68; A61K 45/06; G01N 33/50; G01N 33/53

(52) U.S. Cl. CPC G01N 33/6896; A61K 45/06; G01N 33/5005; G01N 33/53; G01N 33/6881; G01N 2800/2821; G01N 2800/50

(57) ABSTRACT

The invention relates to skin biomarkers, and in particular, to skin biomarkers for diagnosing and prognosing neurodegenerative disorders, such as Alzheimer's disease and Parkinson's disease, as well as diagnostic and prognostic methods and kits for these conditions. The invention also provides methods of treating neurodegenerative disorders. The invention further provides the use of biomarkers in the skin for skin aging (biological & chronological), and kits for detecting and quantifying skin aging, and also methods for treating, preventing or slowing down skin aging.

The invention relates to skin biomarkers, and in particular, to skin biomarkers for diagnosing and prognosing neurodegenerative disorders, such as Alzheimer's disease and Parkinson's disease, as well as diagnostic and prognostic methods and kits for these conditions. The invention also provides methods of treating neurodegenerative disorders. The invention further provides the use of biomarkers in the skin for skin aging (biological & chronological), and kits for detecting and quantifying skin aging, and also methods for treating, preventing or slowing down skin aging.

Alzheimer's disease primarily affects men and women over the age of 65 and the likelihood of being diagnosed with the disease increases substantially with age. With the percentage of adults over the age of 65 expected to grow worldwide over the next 40 years, the incidence of Alzheimer's disease is expected to more than double, escalating from 21 million cases in 2010 to 53 million in 2050 (statistics from www.alzheimersresearchuk.org and www.alz.org). This exponential increase in the expected number of patients presenting with Alzheimer's disease not only represents a major area of unmet medical need, but offers a significant market opportunity for therapeutics and diagnostics as there is currently no fully effective method of treating the disease.

Acetylcholinesterase (AChE) is expressed at different stages of development in various forms, all of which have identical enzymatic activity, but which have very different molecular composition. The ‘tailed’ (T-AChE-SEQ ID No: 1) is expressed at synapses and the inventors have previously identified two peptides that could be cleaved from the C-terminus, one referred to as “T14” (SEQ ID No: 3), within the other which is known as “T30” (SEQ ID No: 2), and which both have strong sequence homology to the comparable region of β-amyloid. The AChE C-terminal peptide “T14”′ has been identified as being the salient part of the AChE molecule responsible for its range of non-hydrolytic actions.

The synthetic 14 amino acids peptide analogue (i.e. “T14”), and subsequently the larger, more stable, and more potent amino acid sequence in which it is embedded (i.e. “T30”) display actions comparable to those reported for ‘non-cholinergic’ AChE, whereas the inert residue within the T30 sequence (i.e. “T15”-SEQ ID No: 4) is without effect. The T14 peptide binds to an allosteric site on the α7 nicotinic-receptor, where, on its own, it has no effect. However, in the presence of a primary ligand, such as acetylcholine or dietary choline, T14 enhances the calcium influx induced by these primary agents. Excessive calcium can be taken up into the mitochondria where it compromises oxidative phosphorylation, and causes a leakage of electrons. Free radicals are consequently formed that then destabilize the cell membrane, and the cell then dies.

T14 is a biomarker of cellular development and growth, and the inventors have previously shown that T14 peptide levels are increased in the brain and cerebrospinal fluid (CSF) of Alzheimer's patients, due to excessive and inappropriate neuronal growth. As such, in their previous work, the inventors demonstrated that T14, in brain tissue or CSF, may be used as a robust diagnostic biomarker for early detection of neurodegenerative disease. In particular, WO 2016/156803 describes an antibody raised against T14, which can selectively recognise and quantify this innate signalling molecule in either human and rat brain tissue or blood samples, by ELISA or Western blotting techniques.

However, accessing nervous tissue for the purposes of biomarker acquisition presents numerous difficulties, and therefore, the current methods of diagnosis and prognosis for Alzheimer's disease are associated with several challenges. So far, many of the efforts towards a biomarker have focused on accessing peripheral fluid tissues that are routinely collected as part of prospective studies. Primarily, serum and plasma are the most available and routinely collected, while saliva and urine are sometimes available from commercial and academic biobanks. However, the complexities of working with plasma as a matrix in the context of measuring a central nervous system (CNS) peptide are numerous, including the association with plasma proteins or metabolites and concomitant lack of free analyte in the blood, matrix interference, protein agglomeration, retention of analyte in the CNS and non-representation in the periphery.

Additionally, a further disadvantage associated with the analysis of biomarkers in samples such as CSF or blood, are that they often require more complex and invasive sample collection from the patient.

There is, therefore, a need to provide improved methods and kits for prognosing and diagnosing patients with neurodegenerative disorders, and especially Alzheimer's disease.

Accordingly, the inventors have continued their previous work in this area, and have focused on the toxic peptide, T14. The inventors have now surprisingly demonstrated that the T14 peptide (SEQ ID No: 3) can be detected in skin, which was unexpected, and even more surprisingly, have discovered that T14 expression correlates with age in that it significantly decreases with age. Accordingly, these two surprising observations demonstrate that the T14 peptide in skin will have significant utility as a biomarker for both skin aging, as well as in the diagnosis and prognosis of neurodegenerative disorders, such as Alzheimer's disease or Parkinson's disease, given the inventors' previous work on the correlation between the T14 peptide and neurodegenerative disease. Therefore, the invention described herein provides significant advantages over previous methods of diagnosis and prognosis, given that skin samples can be readily obtained using methods that are much easier, quicker and less invasive compared to methods required to obtain a sample from the CSF or blood.

Therefore, according to a first aspect of the invention, there is provided the use of a peptide of SEQ ID No: 3, or a variant or fragment thereof, as a diagnostic or prognostic skin biomarker for a neurodegenerative disorder.

As mentioned above, the inventors have previously demonstrated in WO 2016/156803 that T14 peptide levels are increased in the brain tissue and CSF of Alzheimer's patients, when compared to healthy controls. Additionally, as discussed in the Examples, the inventors have now shown that the T14 peptide (SEQ ID No: 3) is surprisingly detectable in the skin, and that T14 peptide levels are high in young, growing skin, whereas T14 peptide levels decrease in older, aged skin. As such, this suggests that an increased level of T14 peptide observed in old skin compared to a healthy control, will indicate that the subject suffers from a neurodegenerative disease.

Therefore, in a second aspect of the invention, there is provided a method for diagnosing a subject who suffers from a neurodegenerative disorder, or a pre-disposition thereto, or for providing a prognosis of the subject's condition, the method comprising analysing, in a skin sample obtained from the subject, the concentration of a peptide of SEQ ID No: 3, or a variant or fragment thereof, and comparing this concentration with a reference for the concentration of a peptide of SEQ ID No: 3, or a variant or fragment thereof in a subject who does not suffer from a neurodegenerative disease, wherein an increase in the concentration of the peptide of SEQ ID No: 3, or a variant or fragment thereof, suggests that the subject suffers from a neurodegenerative disorder, or has a pre-disposition thereto, or the subject's condition has a negative prognosis.

It was unexpected that the T14 peptide (SEQ ID No: 3) would be detectable in the skin at all.

Thus, in a third aspect, there is provided a method for detecting SEQ ID No: 3, or a variant or fragment thereof in a test subject, the method comprising detecting the concentration of the SEQ ID No: 3, or a variant or fragment thereof in a skin sample from the subject.

The invention also provides a kit for diagnosing patients suffering from neurodegenerative disease by detecting the T14 peptide in a skin sample.

Hence, according to a fourth aspect of the invention, there is provided a kit for diagnosing a subject suffering from a neurodegenerative disorder, or a pre-disposition thereto, or for providing a prognosis of the subject's condition, the kit comprising detection means for determining the concentration of a peptide of SEQ ID No: 3, or a variant or fragment thereof, present in a skin sample from a test subject, and a reference for the concentration of a peptide of SEQ ID No: 3, or a variant or fragment thereof in a subject who does not suffer from a neurodegenerative disease, wherein an increase in the concentration of the peptide of SEQ ID No: 3, or a variant or fragment thereof, in the skin sample suggests that the subject suffers from a neurodegenerative disorder, or a pre-disposition thereto.

Preferably, the concentration of a peptide of SEQ ID No: 3, or a variant or fragment thereof, is analysed by a non-invasive method. Preferably, the concentration of a peptide of SEQ ID No: 3, or a variant or fragment thereof, is analysed by immunohistochemistry. Thus, preferably, the detection means of the method and/or kit comprises the use of immunohistochemistry.

Preferably, the neurodegenerative disorder is selected from a group consisting of Alzheimer's disease; Parkinson's disease; Huntington's disease; Motor Neurone disease; Spinocerebellar type 1, type 2, and type 3; Amyotrophic Lateral Sclerosis (ALS); and Frontotemporal Dementia. Most preferably, the neurodegenerative disorder is Alzheimer's disease.

By way of example, the increase in concentration of SEQ ID No: 3, or a variant or fragment thereof compared to the reference concentration may be at least 10%, preferably at least a 20% increase, more preferably at least a 30% increase, even more preferably at least a 40% increase, and most preferably an increase of at least 50% compared to the reference value concentration. Such increases in the concentration of SEQ ID No: 3, or a variant or fragment thereof infer that the test subject suffers from a neurodegenerative disorder, or a pre-disposition thereto.

Advantageously, the use, methods and kit of the invention, as described herein, enable the diagnosis or prognosis of a subject who suffers from a neurodegenerative disorder, or has a pre-disposition thereto. The inventors have found that the T14 peptide (SEQ ID No: 3, or a variant or fragment thereof) is a robust biomarker for identifying subjects who suffer from neurodegenerative disease. Thus, subjects diagnosed with a neurodegenerative disorder, or who have a predisposition thereto, or have a negative prognosis, according to the use, methods and kit of the invention, can benefit from the early therapeutic treatment in order to prevent the onset of a neurodegenerative disorder.

Therefore, methods of the invention may comprise administering or having administered, to the subject, a therapeutic agent or putting the subject on a specialised diet, wherein the therapeutic agent or the specialised diet prevents, reduces or delays progression of the neurodegenerative disorder.

Thus, in a fifth aspect, there is provided a method of treating a subject suffering from a neurodegenerative disorder, said method comprising:
(i) analysing, in a skin sample obtained from the subject, the concentration of a peptide of SEQ ID No: 3, or a variant or fragment thereof, and comparing this concentration with a reference for the concentration of a peptide of SEQ ID No: 3, or a variant or fragment thereof in a subject who does not suffer from a neurodegenerative disease, wherein an increase in the concentration of the peptide of SEQ ID No: 3, or a variant or fragment thereof, suggests that the subject suffers from a neurodegenerative disorder, or has a pre-disposition thereto, or the subject's condition has a negative prognosis; and
(ii) administering or having administered, to the test subject, a therapeutic agent or putting the test subject on a specialised diet, wherein the therapeutic agent or the specialised diet prevents, reduces or delays progression of a neurodegenerative disorder.

Suitable therapeutic agents for treating neurodegenerative disorders may include cyclic polypeptides, such as those disclosed in WO 2015/004430, or linear polypeptides, such as those disclosed in WO 2015/053601. Anti-T14 antibodies, such as those disclosed in WO 2016/156803, may be also used as therapeutic agents. Alternatively, suitable therapeutic agents may include acetylcholinesterase inhibitors, such as Rivastigmine, Galanthamine, and Donepezil, and/or N-methyl-D-aspartate (NMDA) antagonists, such as Memantine.

Suitable specialised diets for treating neurodegenerative disorders may include a Keto diet and/or a Mediterranean diet. Additionally, suitable specialised diets may include vitamin E, vitamin D, β-group vitamins, polyphenols, carotenoids, capsaicin, n-3 polyunsaturated fatty acids (PUFAs), and/or monounsaturated fatty acids (MUFAs).

Preferably, a skin sample is taken from the subject every 13 months, 15 months or 18 months and the concentration of a peptide of SEQ ID No: 3, or a variant or fragment thereof, is compared with the reference for the concentration of a peptide of SEQ ID No: 3, or a variant or fragment thereof in a subject who does not suffer from a neurodegenerative disease. Preferably, a skin sample is taken from the subject every ten, eleven or 12 months and the concentration of a peptide of SEQ ID No: 3, or a variant or fragment thereof, is compared with the reference. Preferably, a skin sample is taken from the subject every six, seven, eight or nine months and the concentration of a peptide of SEQ ID No: 3, or a variant or fragment thereof, is compared with the reference. Preferably, a skin sample is taken from the subject every two, three, four or five months and the concentration of a peptide of SEQ ID No: 3, or a variant or fragment thereof, is compared with the reference. Preferably, a skin sample is taken from the subject every month and the concentration of a peptide of SEQ ID No: 3, or a variant or fragment thereof, is compared with the reference.

It will be appreciated that the concentration of T14 peptide in patients suffering from a neurodegenerative disease is highly dependent on a number of factors, for example how far the disease has progressed, and the age and gender of the subject. It will also be appreciated that the reference concentration of T14 peptide in subjects who do not suffer from the neurodegenerative disease may fluctuate to some degree, but that on average over a given period of time, the concentration tends to be substantially constant.

In addition, it should be appreciated that the concentration of T14 peptide in one group of subjects who do not suffer from a neurodegenerative disease may be different to the concentration of T14 peptide in another group of subjects who do not suffer from the neurodegenerative disease. However, it is possible to determine the average concentration of T14 peptide in subjects who do not suffer from the neurodegenerative disease, and this is referred to as the reference or ‘normal’ concentration of T14 peptide. The normal concentration corresponds to the reference values discussed above.

Furthermore, as described in the Examples, the inventors surprisingly demonstrated that T14 peptide levels were reduced in old skin tissue (53, 54 and 56 years) when compared to young skin tissue (16, 23 and 29 years). Additionally, the inventors also demonstrated that T14 expression decreased in age-matched photo-exposed skin compared to photo-protected skin. As such, these data suggest that the T14 peptide can also be used as a biomarker in the skin for skin aging. In other words, this means that by detecting T14 peptide levels in the skin, an individual is able to compare their biological skin age with their chronological age. Biological age (or physiological age) is the age a subject's body resembles or functions at, and is influenced by various factors such as diet, exercise, stress, exposure to environmental and other toxins, and chronic conditions. However, chronological age is the amount of time (i.e. years, months, days) that have actually passed from a subject's birth to a given date. For example, biological and chronological aging are discussed in more detail in Waaijer M E, Gunn D A, Catt S D, et al. Morphometric skin characteristics dependent on chronological and biological age: the Leiden Longevity Study. Age (Dordr). 2012; 34 (6): 1543-1552. doi: 10.1007/s11357-011-9314-5. Characteristics of biological and chronological skin aging include thin and dry skin, fine wrinkles, decreased elasticity, aberrant pigmentation, increased blemishes, hair greying and hair loss. Advantageously, therefore, if an individual's biological skin age is older than their chronological age, they can apply cosmetic treatments, such as moisturisers or anti-aging creams, as well as change their lifestyle habits, such as by avoiding sunbathing or smoking or excessive alcohol intake, in order to delay or reverse the progression of skin aging.

Accordingly, in a sixth aspect of the invention, there is provided the use of a peptide of SEQ ID No: 3, or a variant or fragment thereof, in skin as a biomarker for skin aging.

In a seventh aspect, there is provided a method for detecting skin aging in a subject, the method comprising analysing, in a skin sample obtained from the subject, the concentration of a peptide of SEQ ID No: 3, or a variant or fragment thereof, and comparing this concentration with a reference for the concentration of a peptide of SEQ ID No: 3, or a variant or fragment thereof in a younger skin sample wherein a decrease in the concentration of the peptide of SEQ ID No: 3, or a variant or fragment thereof, suggests aging of the subject's skin.

In some embodiments, the method comprises detecting biological skin aging in a subject. In some embodiments, the method comprises detecting chronological skin aging in a subject. Accordingly, in this embodiment, a decrease in the concentration of the peptide of SEQ ID No: 3, or a variant or fragment thereof compared to the younger (i.e. the actual chronological age) skin sample, suggests biological aging of the subject's skin.

Preferably, the method comprises quantifying the age of a subject's skin. Even more preferably, the method comprises quantifying the biological age of a subject's skin. In some embodiments, the method comprises comparing the biological age of the subject's skin to their chronological age. Accordingly, an older biological skin age compared to the subject's chronological age suggests aging of the subject's skin.

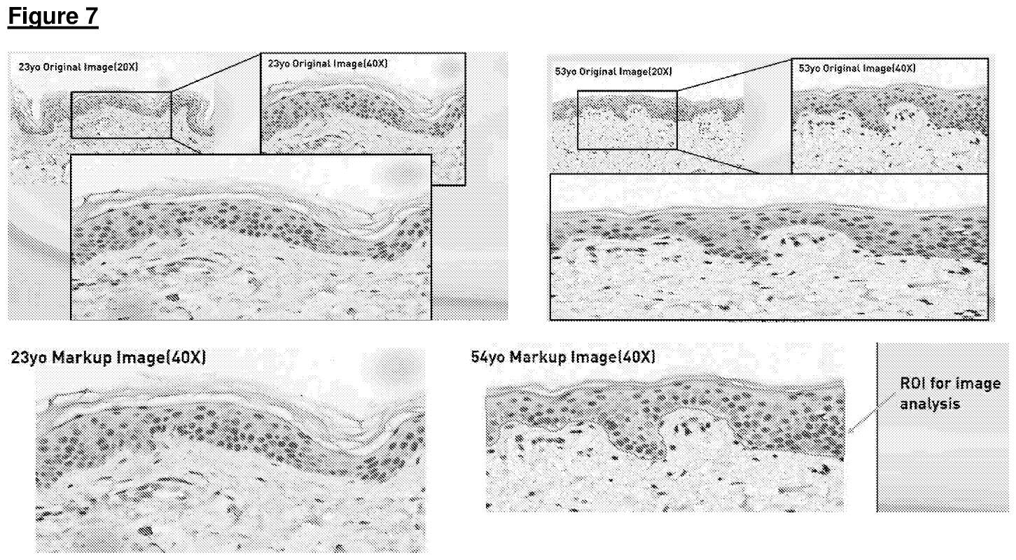

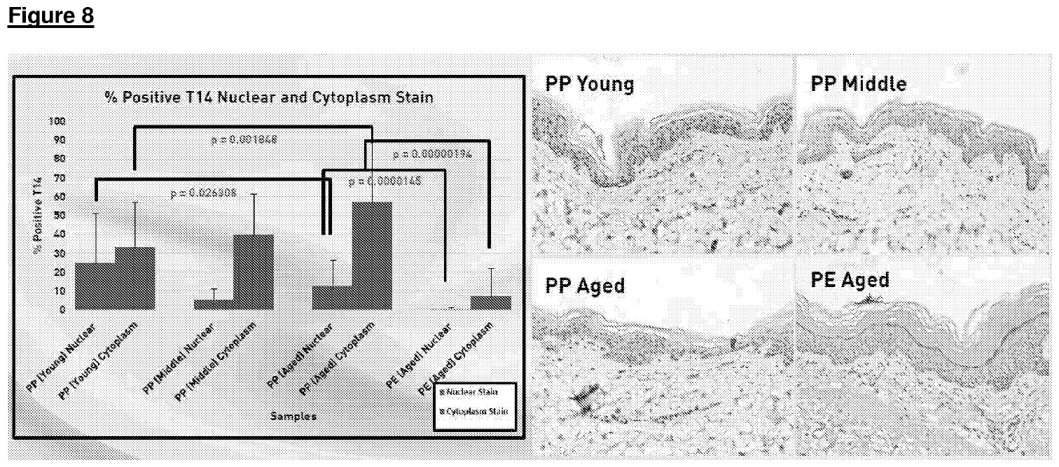

As illustrated in FIGS. 7 and 8, the inventors also discovered that T14 is mainly localised in the nuclei of epidermal cells in younger skin samples, and this localisation in the nuclei decreases with age. Additionally, the inventors observed that T14 localisation in the cytoplasm increases in aged photo-protected skin samples, but decreases in aged photo-exposed skin samples.

Accordingly, in one embodiment, the method comprises analysing the concentration of a peptide of SEQ ID No: 3, or a variant or fragment thereof, in the nuclei of epidermal cells in the skin sample. In this embodiment, a decrease in the concentration of the peptide of SEQ ID No: 3, or a variant or fragment thereof in the nuclei of epidermal cells in the skin sample compared to the younger skin sample, suggests aging of the subject's skin. Preferably, in this embodiment, the aging is biological and/or chronological aging.

In another embodiment, the method comprises analysing the concentration of a peptide of SEQ ID No: 3, or a variant or fragment thereof, in the cytoplasm of epidermal cells in the skin sample. In this embodiment, an increase in the concentration of the peptide of SEQ ID No: 3, or a variant or fragment thereof in the cytoplasm of epidermal cells in the skin sample compared to the younger skin sample, suggests aging of the subject's skin, preferably chronological aging. Alternatively, a decrease in the concentration of the peptide of SEQ ID No: 3, or a variant or fragment thereof in the cytoplasm of epidermal cells in the skin sample compared to the younger skin sample, suggests aging of the subject's skin, preferably biological aging.

In an eighth aspect of the invention, there is provided a kit for detecting skin aging in a subject, the kit comprising detection means for determining the concentration of a peptide of SEQ ID No: 3, or a variant or fragment thereof, present in a skin sample from a test subject, and a reference for the concentration of a peptide of SEQ ID No: 3, or a variant or fragment thereof in a younger skin sample wherein a decrease in the concentration of the peptide of SEQ ID No: 3, or a variant or fragment thereof, in the skin sample suggests aging of the subject's skin.

In some embodiments, the kit comprises means for detecting biological aging in a subject. Accordingly, a decrease in the concentration of the peptide of SEQ ID No: 3, or a variant or fragment thereof compared to the younger skin sample, suggests biological aging of the subject's skin.

Preferably, the kit comprises means for quantifying the age of a subject's skin. Even more preferably, the kit comprises means for quantifying the biological age of a subject's skin. Preferably, the kit comprises means for quantifying the chronological age of a subject's skin. In some embodiments, the kit comprises means for comparing the biological age of the subject's skin to their chronological age. Accordingly, an older biological skin age compared to the subject's chronological age suggests aging of the subject's skin.

Preferably, the concentration of a peptide of SEQ ID No: 3, or a variant or fragment thereof, is analysed by immunohistochemistry. Thus, preferably, the detection means of the method and/or kit comprises the use of immunohistochemistry.

Methods of the invention may comprise administering or having administered, to the subject, a cosmetic agent, a therapeutic agent or putting the subject on a specialised diet, wherein the cosmetic or therapeutic agent or the specialised diet prevents, reduces or delays or reverses progression of skin aging. Additionally, methods of the invention may comprise changes to an individual's lifestyle, such that they avoid activities, such as smoking, sunbathing, and drinking alcohol, which can all accelerate the skin aging process.

Thus, in a ninth aspect, there is provided a method of treating the skin of a subject, said method comprising:
(i) analysing, in a skin sample obtained from the subject, the concentration of a peptide of SEQ ID No: 3, or a variant or fragment thereof, and comparing this concentration with a reference for the concentration of a peptide of SEQ ID No: 3, or a variant or fragment thereof in a younger skin sample wherein a decrease in the concentration of the peptide of SEQ ID No: 3, or a variant or fragment thereof, suggests aging of the subject's skin; and
(ii) administering or having administered, to the test subject, a cosmetic agent, or a therapeutic agent or putting the test subject on a specialised diet, lifestyle or treatment regimen, wherein the cosmetic or therapeutic agent or the specialised diet, lifestyle or treatment regimen prevents, reduces or delays progression of skin aging.

In some embodiments, the cosmetic agent may be a suntan cream, oil or lotion.

In other embodiments, the cosmetic agent may be a moisturiser or moisturising formulation. In other embodiments, the cosmetic agent may be an anti-aging or anti-wrinkle formulation.

Preferably, the cosmetic agent may be administered topically in the form of a cream, gel, lotion, ointment, cutaneous solution, suspension, spray, foam, bath additive, collodion, impregnated dressing or medicated plaster. The cream may be either oil-in-water or water-in-oil type. The cosmetic agent may be administered topically with emulgators such as alkyl sulphates, alkyl amines, alkyl pyrimidin compounds, etc. Acceptable oils for cream formulation include: white petrolatum, paraffin, cetearyl alcohol, cocoglycerides, cetyl alcohol, isopropyl miristate, cetyl palmitate, butyrum cacao, oleum helianthi, cera alba, lanolin, isopropyl palmitate, stearic acid, magnesium stearate. For preparation of a gel, the following gel forming additives may be used: cellulose gum (carboxymethyl cellulose), hydroxypropyl cellulose, methylcellulose, hydroxyethyl cellulose, ethylhydroxy cellulose, or laponite.

Preferably, the cosmetic agent may be administered topically with preserving agents, antioxidants, complexing agents, solvents, fragrances, bactericides, odour absorbers, vitamins, moisturizers, self-tanning compounds and anti-wrinkle active agents.

Suitable therapeutic agents for treating skin aging may include retinoids, vitamin C and/or alpha hydroxy acids.

Suitable specialised diets for treating skin aging may include antioxidant-rich fruit and vegetables, and/or healthy fats, such as from oily fish and nuts. A suitable specialised diet may be a varied and balanced diet, which is one that gives optimal levels of the nutrients that are required for healthy/radiant skin, including beta carotene, vitamin C, vitamin E, zinc and/or selenium.

Suitable lifestyles for treating skin aging may include avoiding activities such as sunbathing, limiting exposure to the sun, and wearing protective clothing and/or suntan lotion. Additionally, lifestyle changes may include avoiding smoking or drinking alcohol, and/or having earlier nights.

Suitable treatment regimens for treating skin aging may include chemical peels, dermabrasion, microdermabrasion, microneedling, and/or laser resurfacing.

Preferably, the reference for the concentration of a peptide of SEQ ID No: 3, or a variant or fragment thereof in a younger skin sample is taken from the same subject as the test sample, but at an earlier time point. Preferably, the younger skin sample is taken from the subject at least one month before the test sample, such that a direct comparison of the concentration of the peptide of SEQ ID No: 3, or a variant or fragment thereof can be made between the test sample and the younger (i.e. chronologically speaking) reference skin sample. Preferably, the younger skin sample is taken from the subject at least two, three or four months before the test sample. Preferably, the younger skin sample is taken from the subject at least five, six, seven or eight months before the test sample. Preferably, the younger skin sample is taken from the subject at least nine, 10, 11 or 12 months before the test sample.

Preferably, at least two, three or four skin samples are taken from the subject at spaced apart times for comparison purposes such that the skin age (biological and/or chronological age) can be monitored over time. Preferably, a skin sample is taken from the subject every ten, eleven or 12 months and the concentration of a peptide of SEQ ID No: 3, or a variant or fragment thereof, is compared with the concentration in the younger reference skin sample. Preferably, a skin sample is taken from the subject every six, seven, eight or nine months and the concentration of a peptide of SEQ ID No: 3, or a variant or fragment thereof, is compared with the concentration in the younger reference skin sample. Preferably, a skin sample is taken from the subject every two, three, four or five months and the concentration of a peptide of SEQ ID No: 3, or a variant or fragment thereof, is compared with the concentration in the younger reference skin sample. Preferably, a skin sample is taken from the subject every month or week and the concentration of a peptide of SEQ ID No: 3, or a variant or fragment thereof, is compared with the concentration in the younger reference skin sample.

By way of example, the decrease in concentration of SEQ ID No: 3, or a variant or fragment thereof (i.e. the T14 peptide) compared to the reference concentration may be at least 10%, preferably at least a 20% decrease, more preferably at least a 30% decrease, even more preferably at least a 40% decrease, and most preferably a decrease of at least 50% from the reference value concentration. Such decreases in SEQ ID No: 3, or a variant or fragment thereof (T14 peptide) concentrations infer biological aging of the test subject's skin.

Acetylcholinesterase is a serine protease that hydrolyses acetylcholine, and is well-known to the skilled person. The major form of acetylcholinesterase, which is found in the brain is known as tailed acetylcholinesterase (T-AChE). The protein sequence of one embodiment of human tailed acetylcholinesterase (Gen Bank: AAA68151.1) is 614 amino acids in length, and is provided herein as SEQ ID No: 1, as follows:

```
[SEQ ID No: 1]
    1 mrppqcllht pslaspllll llwllgggvg aegredaell vtvrggrlrg irlktpggpv
  61 saflgipfae ppmgprrflp pepkqpwsgv vdattfqsvc yqyvdtlypg fegtemwnpn
121 relsedclyl nvwtpyprpt sptpvlvwiy gggfysgass ldvydgrflv qaertvlvsm
181 nyrvgafgfl alpgsreapg nvglldqrla lqwvqenvaa fggdptsvtl fgesagaasv
241 gmhllsppsr glfhravlqs gapngpwatv gmgearrrat qlahlvgcpp ggtggndtel
301 vaclrtrpaq vlvnhewhvl pqesvfrfsf vpvvdgdfls dtpealinag dfhglqvlvg
361 vvkdegsyfl vygapgfskd neslisraef lagvrvgvpq vsdlaaeavv lhytdwlhpe
421 dparlreals dvvgdhnvvc pvaqlagrla aqgarvyayv fehrastlsw plwmgvphgy
481 eiefifgipl dpsrnytaee kifaqrlmry wanfartgdp neprdpkapq wppytagaqq
541 yvsldlrple vrrglraqac afwnrflpkl lsatdtldea erqwkaefhr wssymvhwkn
601 qfdhyskqdr csdl
```

It will be appreciated that the first 31 amino acid residues of SEQ ID No: 1 are removed while the protein is released, thereby leaving a 583 amino acid sequence.

The amino acid sequence of T30 (which corresponds to the last 30 amino acid residues of SEQ ID No: 1) is provided herein as SEQ ID No: 2, as follows:—

[SEQ ID No: 2]
KAEFHRWSSYMVHWKNQFDHYSKQDRCSDL

The amino acid sequence of T14 (which corresponds to the 14 amino acid residues located towards the end of SEQ ID No: 1, and lacks the final 15 amino acids found in T30) is provided herein as SEQ ID No: 3, as follows:—

[SEQ ID No: 3]
AEFHRWSSYMVHWK

The amino acid sequence of T15 (which corresponds to the last 15 amino acid residues of SEQ ID No: 1) is provided herein as SEQ ID No: 4, as follows:—

[SEQ ID No: 4]
NQFDHYSKQDRCSDL

The amino acid sequence of part of β-amyloid (Aβ) is provided herein as SEQ ID No: 7, as follows:—

[SEQ ID No: 7]
DAEFRHDSGYEVHHQKLVFFAEDVGSNKGAIIGLMVGGVVIA

Preferably, SEQ ID No: 3, or a variant or fragment thereof acts as an epitope which may be bound by an antibody or antigen-binding fragment used for detecting the T14 peptide in any of the methods, uses and kits of the invention. Thus, the detection means in the kits of the invention is preferably an antibody or antigen binding fragment thereof. For example, WO 2016/156803 describes a suitable antibody which may be used in all aspects of the present invention.

The antibody or antigen-binding fragment thereof may be polyclonal or monoclonal.

The antibody or antigen-binding fragment thereof may be generated in a rabbit, mouse or rat.

Preferably, the antibody or antigen-binding fragment thereof specifically binds to SEQ ID No: 3, or a variant or fragment thereof. Preferably, the antibody or antigen-binding fragment thereof specifically binds to one or more amino acid in the C-terminus of SEQ ID No: 3. Preferably, the antibody or antigen-binding fragment thereof specifically binds to one or more amino acid in SEQ ID No: 5 (i.e. SYMVHWK, which are the C-terminal amino acids numbers 7-14 of SEQ ID No: 3). Preferably, the antibody or antigen-binding fragment thereof specifically binds to a C-terminal lysine (K) residue in the epitope.

The inventors previously observed that the C-terminal amino acid sequence VHWK in SEQ ID No: 3, which is described herein as SEQ ID No. 6 (i.e. the C-terminal amino acids numbers 10-14 of SEQ ID No. 3), acts as an epitope for the antibody or antigen-binding fragment thereof. Accordingly, more preferably the antibody or antigen-binding fragment thereof specifically binds to one or more amino acid in SEQ ID No: 6. Most preferably, the antibody or antigen-binding fragment thereof specifically binds to SEQ ID No: 6. Hence, it will be appreciated that the epitope to which the antibody binds comprises or consists of SEQ ID No: 6.

Preferably, the antibody or antigen-binding fragment thereof does not bind to SEQ ID No: 2. Preferably, the antibody or antigen-binding fragment thereof does not bind to SEQ ID No: 4. Preferably, the antibody or antigen-binding fragment thereof does not bind to β-amyloid (SEQ ID No: 7).

The antibody or antigen-binding fragment thereof may be monovalent, divalent or polyvalent. Monovalent antibodies are dimers (HL) comprising a heavy (H) chain associated by a disulphide bridge with a light chain (L). Divalent antibodies are tetramer (H2L2) comprising two dimers associated by at least one disulphide bridge. Polyvalent antibodies may also be produced, for example by linking multiple dimers. The basic structure of an antibody molecule consists of two identical light chains and two identical heavy chains which associate non-covalently and can be linked by disulphide bonds.

Each heavy and light chain contains an amino-terminal variable region of about 110 amino acids, and constant sequences in the remainder of the chain. The variable region includes several hypervariable regions, or Complementarity Determining Regions (CDRs), that form the antigen-binding site of the antibody molecule and determine its specificity for the antigen, i.e. SEQ ID No: 3, or variant or fragment thereof (e.g. an epitope). On either side of the CDRs of the heavy and light chains is a framework region, a relatively conserved sequence of amino acids that anchors and orients the CDRs. Antibody fragments may include a bi-specific antibody (BsAb) or a chimeric antigen receptor (CAR).

The constant region consists of one of five heavy chain sequences (μ, γ, ζ, α, or ε) and one of two light chain sequences (κ or λ). The heavy chain constant region sequences determine the isotype of the antibody and the effector functions of the molecule.

Preferably, the antibody or antigen-binding fragment thereof is isolated or purified.

Polyclonal antibodies may be produced as polyclonal sera by injecting antigen into animals. Preferred polyclonal antibodies may be raised by inoculating an animal (e.g. a rabbit) with antigen (e.g. T14 or fragments thereof, including the C-terminus) using techniques known to the art. For example, the antibody or antigen-binding fragment thereof may be obtained by immunising a host animal with SEQ ID No: 3, and then collecting the antibody or antigen-binding fragment thereof. The host animal is most preferably a rabbit.

In another preferred embodiment, the antibody or antigen-binding fragment thereof is a monoclonal antibody or an antigen-binding fragment thereof. Preferred monoclonal antibodies may be raised using hybridoma technology using T14 or fragments thereof as antigen. Preferably, the antibody is a human antibody. As used herein, the term “human antibody” can mean an antibody, such as a monoclonal antibody, which comprises substantially the same heavy and light chain CDR amino acid sequences as found in a particular human antibody exhibiting immunospecificity for SEQ ID No: 3, or a variant or fragment thereof. An amino acid sequence, which is substantially the same as a heavy or light chain CDR, exhibits a considerable amount of sequence identity when compared to a reference sequence. Such identity is definitively known or recognizable as representing the amino acid sequence of the particular human antibody. Substantially the same heavy and light chain CDR amino acid sequence can have, for example, minor modifications or conservative substitutions of amino acids. Such a human antibody maintains its function of selectively binding to SEQ ID No: 3, or a variant or fragment thereof.

Conventional hybridoma techniques may be used to raise the antibodies. The antigen used to generate monoclonal antibodies may be the whole T14 protein or a fragment thereof. Preferred fragments for generating the antibodies may also be the peptides discussed above, and particularly SYMVHWK (SEQ ID No: 5) or VHWK (SEQ ID No: 6). It is preferred that the antibody is a γ-immunoglobulin (IgG).

The term “human monoclonal antibody” can include a monoclonal antibody with substantially or entirely human CDR amino acid sequences produced, for example by recombinant methods such as production by a phage library, by lymphocytes or by hybridoma cells.

The term “humanised antibody” can mean an antibody from a non-human species (e.g. mouse or rabbit) whose protein sequences have been modified to increase their similarity to antibodies produced naturally in humans.

The antibody may be a recombinant antibody. The term “recombinant human antibody” can include a human antibody produced using recombinant DNA technology.

The term “antigen-binding region” can mean a region of the antibody having specific binding affinity for its target antigen, for example, the peptide of SEQ ID No: 3, or a variant or fragment thereof. Preferably, the fragment is an epitope. The binding region may be a hypervariable CDR or a functional portion thereof. The term “functional portion” of a CDR can mean a sequence within the CDR which shows specific affinity for the target antigen. The functional portion of a CDR may comprise a ligand which specifically binds to SEQ ID No: 3 or a fragment thereof.

The term “CDR” can mean a hypervariable region in the heavy and light variable chains. There may be one, two, three or more CDRs in each of the heavy and light chains of the antibody. Normally, there are at least three CDRs on each chain which, when configured together, form the antigen-binding site, i.e. the three-dimensional combining site with which the antigen binds or specifically reacts. It has however been postulated that there may be four CDRs in the heavy chains of some antibodies.

The definition of CDR also includes overlapping or subsets of amino acid residues when compared against each other. The exact residue numbers which encompass a particular CDR or a functional portion thereof will vary depending on the sequence and size of the CDR. Those skilled in the art can routinely determine which residues comprise a particular CDR given the variable region amino acid sequence of the antibody.

The term “functional fragment” of an antibody can mean a portion of the antibody which retains a functional activity. A functional activity can be, for example antigen binding activity or specificity. A functional activity can also be, for example, an effector function provided by an antibody constant region. The term “functional fragment” is also intended to include, for example, fragments produced by protease digestion or reduction of a human monoclonal antibody and by recombinant DNA methods known to those skilled in the art. Human monoclonal antibody functional fragments include, for example individual heavy or light chains and fragments thereof, such as VL, VH and Fd; monovalent fragments, such as Fv, Fab, and Fab′; bivalent fragments such as F(ab′)₂; single chain Fv (scFv); and Fc fragments.

The term “VL fragment” can mean a fragment of the light chain of a human monoclonal antibody which includes all or part of the light chain variable region, including the CDRs. A VL fragment can further include light chain constant region sequences.

The term “VH fragment” can means a fragment of the heavy chain of a human monoclonal antibody which includes all or part of the heavy chain variable region, including the CDRs.

The term “Fd fragment” can mean the heavy chain variable region coupled to the first heavy chain constant region, i.e. VH and CH-1. The “Fd fragment” does not include the light chain, or the second and third constant regions of the heavy chain.

The term “Fv fragment” can mean a monovalent antigen-binding fragment of a human monoclonal antibody, including all or part of the variable regions of the heavy and light chains, and absent of the constant regions of the heavy and light chains. The variable regions of the heavy and light chains include, for example, the CDRs. For example, an Fv fragment includes all or part of the amino terminal variable region of about 110 amino acids of both the heavy and light chains.

The term “Fab fragment” can mean a monovalent antigen-binding fragment of a human monoclonal antibody that is larger than an Fv fragment. For example, a Fab fragment includes the variable regions, and all or part of the first constant domain of the heavy and light chains. Thus, a Fab fragment additionally includes, for example, amino acid residues from about 110 to about 220 of the heavy and light chains.

The term “Fab′ fragment” can mean a monovalent antigen-binding fragment of a human monoclonal antibody that is larger than a Fab fragment. For example, a Fab′ fragment includes all of the light chain, all of the variable region of the heavy chain, and all or part of the first and second constant domains of the heavy chain. For example, a Fab′ fragment can additionally include some or all of amino acid residues 220 to 330 of the heavy chain.

The term “F(ab′)₂ fragment” can mean a bivalent antigen-binding fragment of a human monoclonal antibody. An F(ab′)₂ fragment includes, for example, all or part of the variable regions of two heavy chains- and two light chains, and can further include all or part of the first constant domains of two heavy chains and two light chains.

The term “single chain Fv (scFv)” can mean a fusion of the variable regions of the heavy (VH) and light chains (VL) connected with a short linker peptide.

The term “bispecific antibody (BsAb)” can mean a bispecific antibody comprising two scFv linked to each other by a shorter linked peptide.

One skilled in the art knows that the exact boundaries of a fragment of an antibody are not important, so long as the fragment maintains a functional activity. Using well-known recombinant methods, one skilled in the art can engineer a polynucleotide sequence to express a functional fragment with any endpoints desired for a particular application. A functional fragment of the antibody may comprise or consist of a fragment with substantially the same heavy and light chain variable regions as the human antibody.

Preferably, the antigen-binding fragment thereof, is SEQ ID No: 3-specific or immunospecific for an epitope within SEQ ID No: 3. The antigen-binding fragment thereof may comprise or consist of any of the fragments selected from a group consisting of VH, VL, Fd, Fv, Fab, Fab′, scFv, F(ab′)₂ and Fc fragment.

The antigen-binding fragment thereof may comprise or consist of any one of the antigen binding region sequences of the VL, any one of the antigen binding region sequences of the VH, or a combination of VL and VH antigen binding regions of a human antibody.

The appropriate number and combination of VH and VL antigen binding region sequences may be determined by those skilled in the art depending on the desired affinity and specificity and the intended use of the antigen-binding fragment. Functional fragments or antigen-binding fragments of antibodies may be readily produced and isolated using methods well known to those skilled in the art. Such methods include, for example, proteolytic methods, recombinant methods and chemical synthesis. Proteolytic methods for the isolation of functional fragments comprise using human antibodies as a starting material. Enzymes suitable for proteolysis of human immunoglobulins may include, for example, papain, and pepsin. The appropriate enzyme may be readily chosen by one skilled in the art, depending on, for example, whether monovalent or bivalent fragments are required. For example, papain cleavage results in two monovalent Fab′ fragments that bind antigen and an Fc fragment. Pepsin cleavage, for example, results in a bivalent F(ab′) fragment. An F(ab′)₂ fragment of the invention may be further reduced using, for example, DTT or 2-mercaptoethanol to produce two monovalent Fab′ fragments.

Functional or antigen-binding fragments of antibodies produced by proteolysis may be purified by affinity and column chromatographic procedures. For example, undigested antibodies and Fc fragments may be removed by binding to protein A. Additionally, functional fragments may be purified by virtue of their charge and size, using, for example, ion exchange and gel filtration chromatography. Such methods are well known to those skilled in the art.

The antibody or antigen-binding fragment thereof may be produced by recombinant methodology. Preferably, one initially isolates a polynucleotide encoding desired regions of the antibody heavy and light chains. Such regions may include, for example, all or part of the variable region of the heavy and light chains. Preferably, such regions can particularly include the antigen binding regions of the heavy and light chains, preferably the antigen binding sites, most preferably the CDRs.

The polynucleotide encoding the antibody or antigen-binding fragment thereof may be produced using methods known to those skilled in the art. The polynucleotide encoding the antibody or antigen-binding fragment thereof may be directly synthesized by methods of oligonucleotide synthesis known in the art. Alternatively, smaller fragments may be synthesized and joined to form a larger functional fragment using recombinant methods known in the art.

As used herein, the term “immunospecificity” can mean the binding region is capable of immunoreacting with SEQ ID No: 3, or a variant or fragment thereof, by specifically binding therewith. The antibody or antigen-binding fragment thereof can selectively interact with an antigen (e.g. SEQ ID No: 3 or a variant or fragment thereof) with an affinity constant of approximately 10⁻⁵ to 10⁻¹³ M⁻¹, preferably 10⁻⁶ to 10⁻⁹ M⁻¹, even more preferably, 10⁻¹⁰ to 10⁻¹² M⁻¹.

The term “immunoreact” can mean the binding region is capable of eliciting an immune response upon binding with SEQ ID No: 3, or an epitope thereof.

The term “epitope” can mean any region of an antigen with the ability to elicit, and combine with, a binding region of the antibody or antigen-binding fragment thereof.

The subject may be a vertebrate, mammal, or domestic animal. Most preferably, however, the subject is a human being. The subject may be a child or adult. The age of the subject may be at least 20, 25, 30, 35 or 40. The age of the subject may be at least 45, 46, 47, 48, 49 or 50. The age of the subject may be at least 51, 52, 53, 54 or 55. The age of the subject may be at least 56, 57, 58, 59 or 60. The age of the subject may be at least 61, 62, 63, 64 or 65. The age of the subject may be at least 66, 67, 68, 69 or 70.

The methods may be carried out in vivo, in vitro or ex vivo.

Preferably, however, the method is carried out in vitro.

Preferably, the skin sample comprises skin epidermis and/or skin dermis. Preferably, the skin sample comprises stratum basale, stratum spinosum, stratum granulosum, stratum lucidum and/or stratum corneum.

Preferably, the skin sample comprises at least one keratinocyte, and preferably a plurality of keratinocytes. Preferably, the skin sample comprises at least 5, 10, 15 or 20 keratinocytes. Preferably, the skin sample comprises at least 30, 40, 50, 60, 70, 80, 90 or 100 keratinocytes. Preferably, the skin sample comprises at least 150, 200, 250 or 300 keratinocytes.

The skin sample may be obtained from the subject using methods well known to the skilled person in the art. For example, the skin sample may be obtained by a skin punch biopsy. Alternatively, the skin sample may be obtained from scraped cells, skin shave biopsies, skin stripping (e.g. using adhesive tape), or through fluid collected under pressure using reverse iontophoresis.

Alternatively, the skin sample may be obtained using a suction blister method. Briefly, one or more glass chamber may be attached to an area of the skin and connected to a vacuum pump, such that epidermis may be slowly detached from underlying dermis. An induced blister may then be formed that fills with interstitial fluid, which can be extracted with a syringe. Advantageously, the suction blister method is quick, painless and non-invasive.

Thus, preferably the kit comprises means for obtaining the skin sample, which may be a razor, a scalpel, a blade, a biopsy punch and/or adhesive tape. Alternatively, when using the suction blister method, the kit may comprise a glass chamber, a vacuum pump, and/or a syringe.

As mentioned above, preferably the detection means of the method and/or kit comprises the use of immunohistochemistry. Thus, in this embodiment, preferably the kit comprises a slide, hematoxylin, and/or a microscope.

The kit may comprise a sample collection container for receiving the obtained or extracted skin sample.

T14 peptide (SEQ ID No: 3) may be assayed and quantified by a number of ways known to one skilled in the art. For example, preferably, immunoassays may be employed to measure T14 peptide levels. However, it will be appreciated that non-immuno based assays may be employed, for example, labelling a compound having affinity with a ligand of the T14 peptide molecule, and then assaying for the label.

T14 peptide may also be determined with Western Blot analysis. Hence, immunoassays and Western blot analyses may be used to determine the total protein level of T14 peptide. T14 peptide concentration may also be detected by enzyme-linked immunosorbent assay (ELISA), fluorometric assay, chemiluminescent assay, or radioimmunoassay analyses.

The kit may further comprise a label which may be detected. The term “label” can mean a moiety that can be attached to the detection means, or fragment thereof, for example an antibody. Moieties can be used, for example, for therapeutic or diagnostic procedures. Therapeutic labels include, for example, moieties that can be attached to an antibody or fragment thereof and used to monitor the binding of the antibody to T14 peptide (i.e. SEQ ID No: 3). As described herein the antibody or antigen-binding fragment thereof binds specifically to SEQ ID No: 3, or a fragment or variant thereof, and can be used as the detection means, or in the detection means. Preferably, the antibody or antigen-binding fragment thereof does not bind to SEQ ID No: 2 (i.e. T30). Preferably, the antibody or antigen-binding fragment thereof does not bind to SEQ ID No: 4 (i.e. T15). Preferably, the antibody or antigen-binding fragment thereof does not bind to β-amyloid (SEQ ID No: 7).

Diagnostic labels include, for example, moieties which can be detected by analytical methods. Analytical methods include, for example, qualitative and quantitative procedures. Qualitative analytical methods include, for example, immunohistochemistry and indirect immunofluorescence. Quantitative analytical methods include, for example, immunoaffinity procedures such as radioimmunoassay, ELISA or FACS analysis.

Analytical methods also include both in vitro and in vivo imaging procedures. Specific examples of diagnostic labels that can be detected by analytical means include enzymes, radioisotopes, fluorochromes, chemiluminescent markers, and biotin.

It will be appreciated that the invention extends to any nucleic acid or peptide or variant, derivative or analogue thereof, which comprises substantially the amino acid or nucleic acid sequences of any of the sequences referred to herein, including functional variants or functional fragments thereof. The terms “substantially the amino acid/nucleotide/peptide sequence”, “functional variant” and “functional fragment”, can be a sequence that has at least 40% sequence identity with the amino acid/nucleotide/peptide sequences of any one of the sequences referred to herein, for example 40% identity with the sequence identified as SEQ ID No: 1-7, and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence, which hybridizes to DNA sequences or their complements under stringent conditions. By stringent conditions, we mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in SEQ ID No: 1-7.

Due to the degeneracy of the genetic code, it is clear that any nucleic acid sequence described herein could be varied or changed without substantially affecting the sequence of the protein encoded thereby, to provide a functional variant thereof.

Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change.

For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

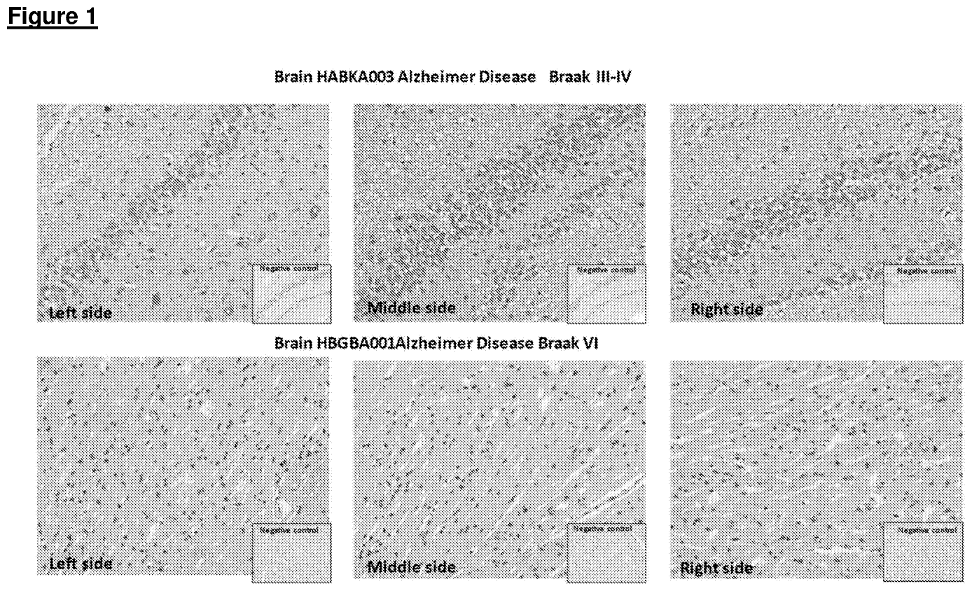

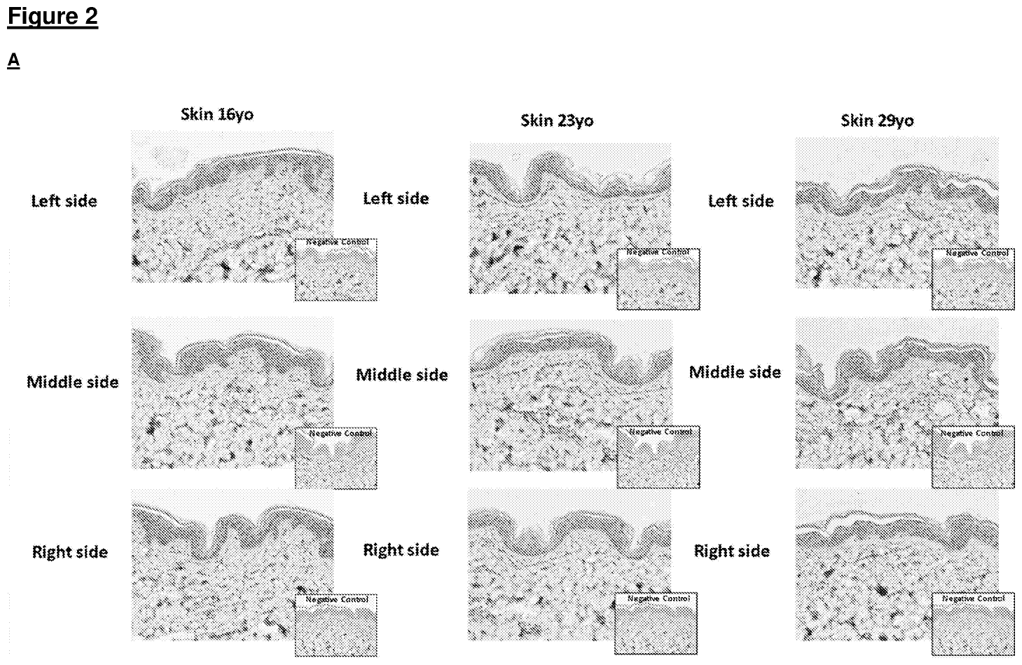

FIG. 1 shows immunohistochemical staining of human Alzheimer's diseased brain tissue in a Braak stage III-IV patient, and in a Braak stage VI patient.

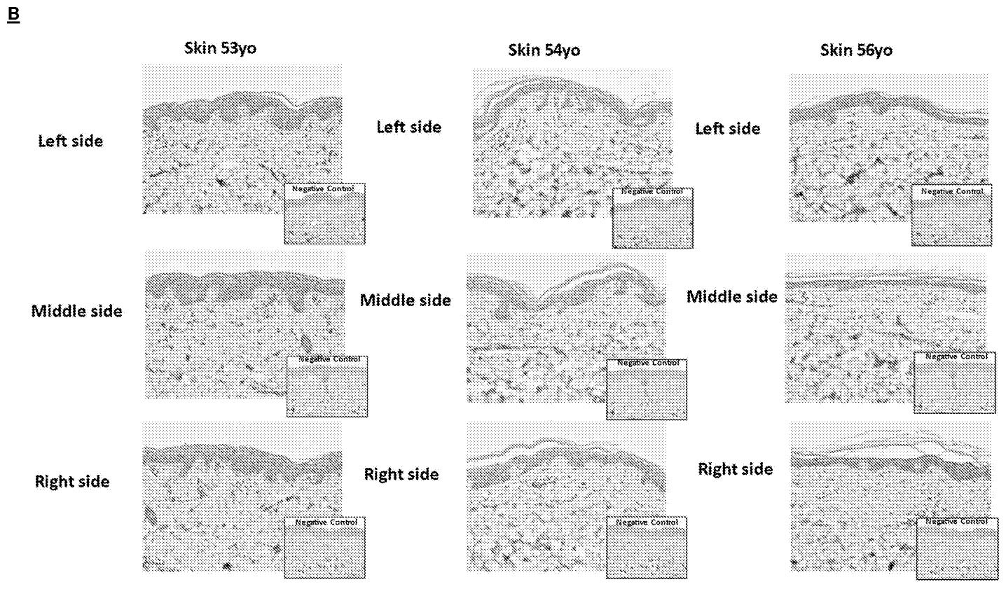

FIG. 2 shows immunohistochemical staining of young (16, 23 and 29 years old) skin tissue (A), and aged (53, 54 and 56 years old) skin tissue (B).

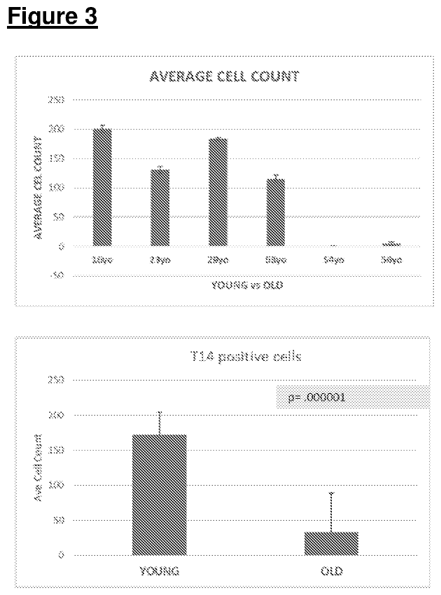

FIG. 3 shows the total average cell count and T14 positive cell count in young (16, 23 and 29 years) and aged (53, 54 and 56 years) skin tissue.

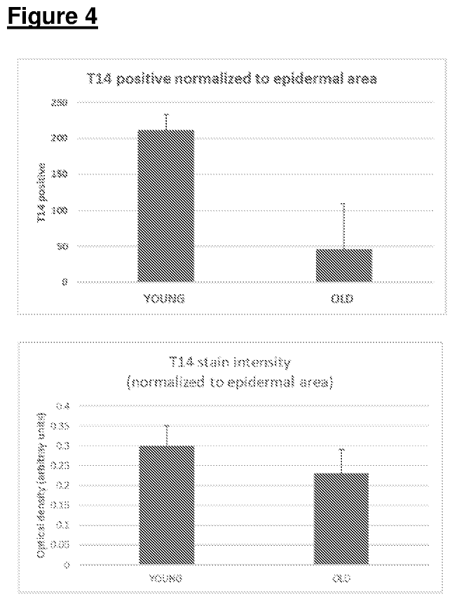

FIG. 4 shows T14 positive cell count and T14 stain intensity normalised to the epidermal area in young (16, 23 and 29 years) and aged (53, 54 and 56 years) skin tissue.

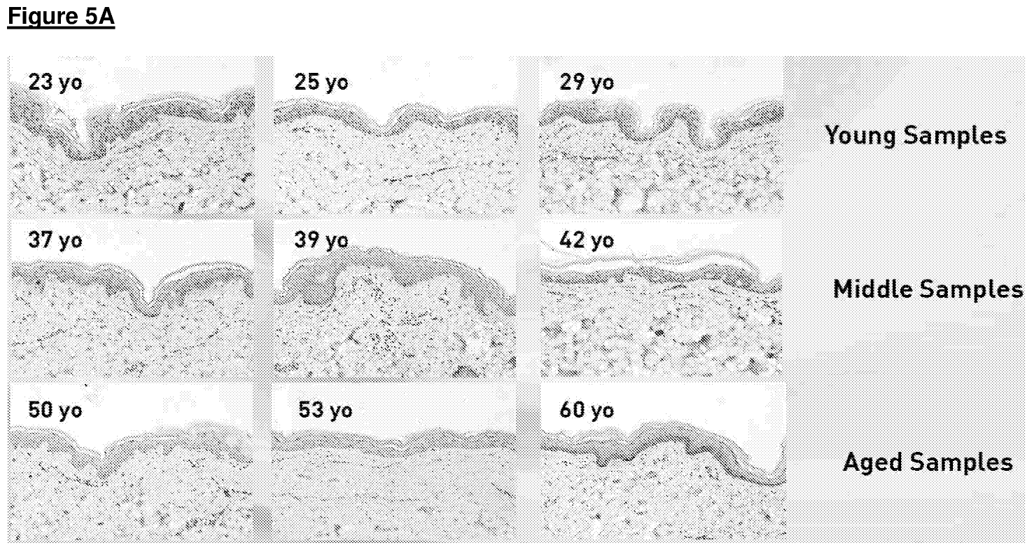

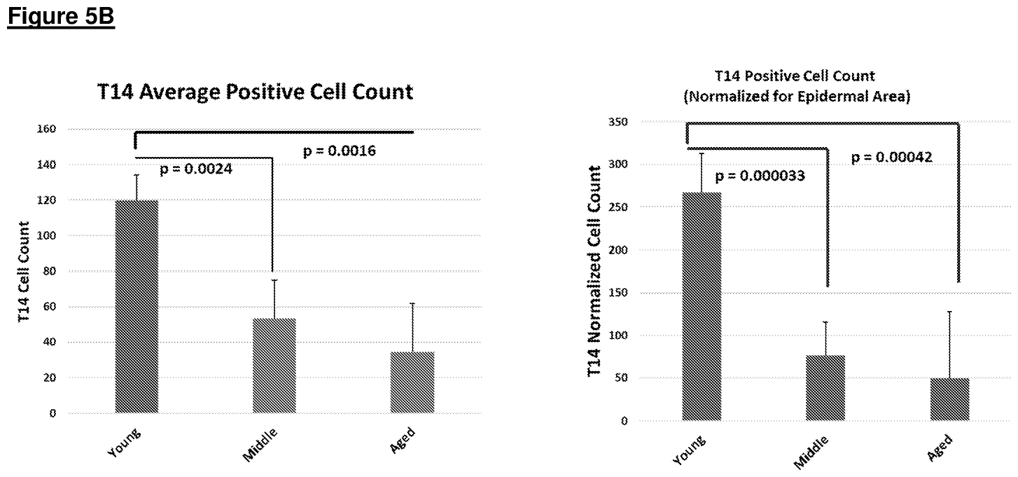

FIG. 5 shows immunohistochemical staining (A) and quantitative results (B) of T14 expression in young, middle and aged skin samples, illustrating that T14 expression and the number of T14 positive cells decreases in aged samples compared to young samples.

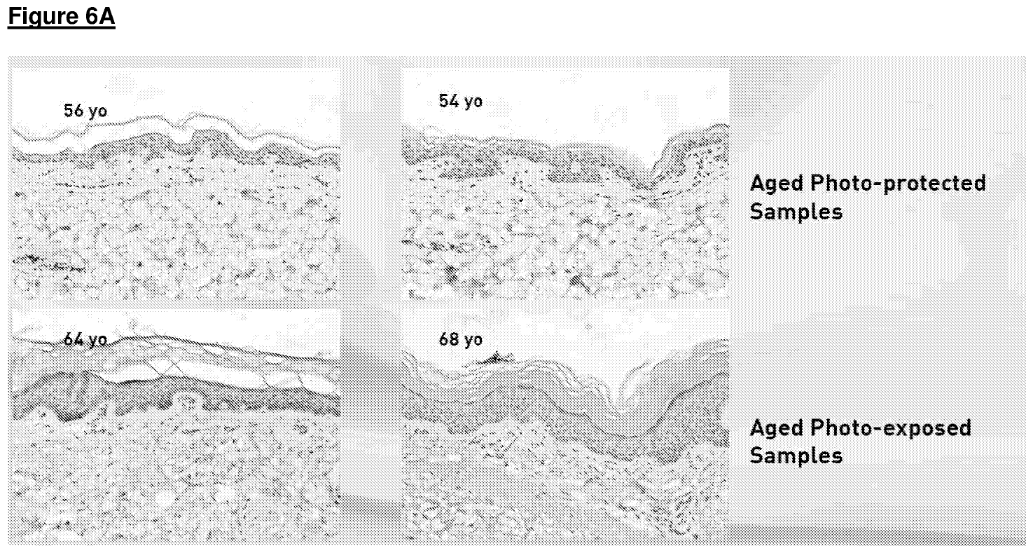

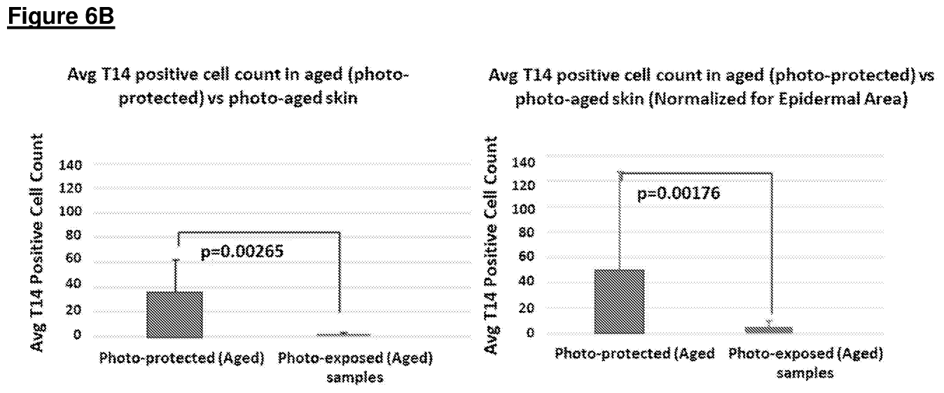

FIG. 6 illustrates the effects of UV-light on levels of T14 in skin. (A) Immunohistochemistry of AChE T14 peptide expression is higher in aged photo-protected (50 to 70 years old) skin compared to aged photo-exposed skin (50 to 70 years old). (B) Average number of epidermal cells with a positive AChE T14 peptide antibody stain. Aged (photo-protected) skin samples have a significantly higher expression (p value of 0.00265) of T14 positive cells compared to its expression in age matched, photo-exposed skin samples. Also shown is the average number of epidermal cells with a positive AChE T14 peptide antibody stain normalized by epidermal area. Aged (photo-protected) skin samples have a significantly higher expression (p value of 0.00176) of T14 positive cells per epidermal area compared to its expression in age-matched photo-exposed skin.

FIG. 7 illustrates immunohistochemical staining showing the localisation of T14 staining in the epidermis. In younger samples, T14 is mainly localised in the nucleus, however, in aged samples, it is also present in the cytoplasm. In the older samples, there was a signal which was possible to differentiate in colour, illustrated as a fainter and more extensive stain, suggestive of a cytoplasmic location.

FIG. 8 illustrates immunohistochemical staining and quantitative T14 expression results in the nucleus and cytoplasm of the epidermis from photo-protected (PP) and photo-exposed (PE) young, middle and aged groups.

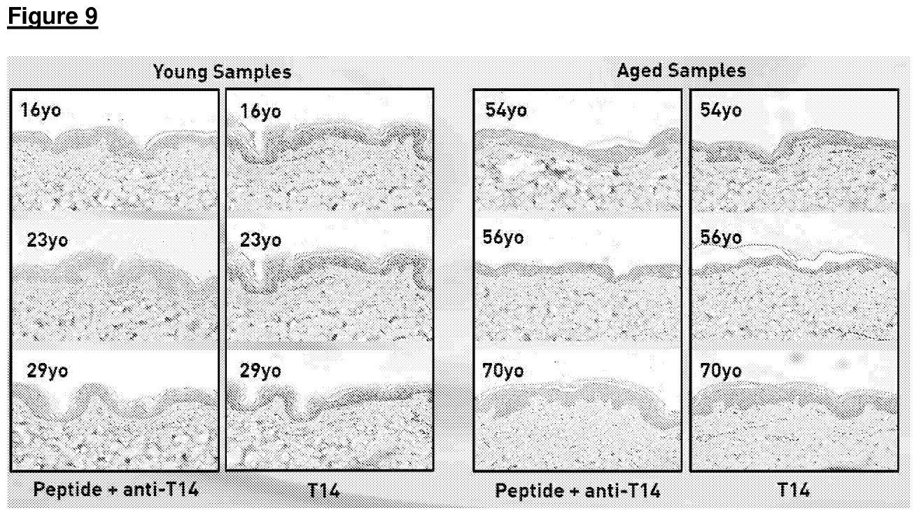

FIG. 9 illustrates immunohistochemical staining in the epidermis after immunoneutralisation of the T14 antibody. As shown, the peptide successfully blocked binding of anti-T14 polyclonal antibody to the T14 epitope for both young and aged skin samples.

### EXAMPLES

### Materials and Methods

### Tissues

Samples consisted of formalin-fixed paraffin embedded (FFPE) skin and brain tissues. Young (16, 23 and 29 years old) and aged (53, 54 and 56 years old) skin tissue were procured from BioIVT; Alzheimer's diseased brain tissue (grades Braak III-IV and VI) were obtained from Tissue Solutions (Glasgow, UK).

An extended skin panel was also made up of photo-protected and photo-exposed skin samples (10 subjects per group).

Photo-protected skin comprised the following tissue samples:
Young: 16-29 years old (abdominal or breast skin)
Middle age: 40-50 years old (abdominal or breast skin)
Aged: 50+ years old (abdominal or breast skin).

Photo-exposed skin comprised the following tissue samples:
48+ years old (face, arm or back skin).

### Immunohistochemistry (IHC)

Immunohistochemical staining was performed using standard techniques. Briefly, each sample was sectioned at a thickness of 5 μm and multiple sections were placed on each slide. Slides were cleared and rehydrated prior to IHC being performed. High heat epitope retrieval (Diva Decloaker 10× DV2004MX Biocare Medical) was performed in a decloaking chamber (Biocare Medical), then slides were washed in tris-buffered saline (TBS). The subsequent steps were all performed using an IntelliPath immunostainer (Biocare Medical), including blocking steps IP peroxide (PX968MM Biocare Medical), block for 5 minutes followed by 1 minute wash in buffer (TBS auto wash buffer 20× TWB945M Biocare Medical). Background Sniper (BS966MM Biocare Medical) block for 10 minutes followed by 1 minute wash in buffer (TBS auto wash buffer20× TWB945M Biocare Medical). Primary antibody incubation (1:100 dilution of rabbit anti-T14 (Genosphere Biotechnologies), primary antibody dilution buffer (Da Vinci Green Diluent PD900M Biocare Medical), Rabbit Mach 4 AP Polymer (MRAP536L Biocare Medical) for 10 minutes followed by 1 minute wash in buffer (TBS auto wash buffer20× TWB945M Biocare Medical). IP Fast Red Chromogen (IPK5017G80 Biocare Medical) for 15 minutes followed by 1 minute wash in buffer (TBS auto wash buffer 20× TWB945M Biocare Medical). Counterstain with Hematoxylin (Tacha's Auto Hematoxylin cat #NM-HEM-M Biocare Medical) for 3 minutes. Sections were then dehydrated in alcohol washes, run through xylene washes, and cover slipped.

Alzheimer's diseased brain tissue (grades Braak III-IV and VI) and skin samples (23 and 53 years old) were used as negative controls, in which no primary antibody (anti-T14 antibody) was added. Negative controls are illustrated in the inset panels of FIGS. 1 and 2.

### Image Acquisition and Analysis

Image acquisition was performed on a Zeiss Axioplan 2 microscope with a Nuance multispectral camera. All quantitative analysis was performed using the HALO image analysis platform (Indica Labs).

### Nuclear and Cytoplasm Staining Analysis

### Nuclear Analysis:

% Positive Stain-Quantification of number of T14 positive cells normalized against total number of cells in the viable epidermis.

### Cytoplasm Analysis:

% Positive Stain—Percentage of keratinocytes with cytoplasm stain

### Parameter Settings are Adjusted for Nuclear and Cytoplasm Quantitation:

Nuclear Contrast Threshold
Minimum Nuclear Intensity
Nuclear Segmentation Aggressiveness
Nuclear Size and Nuclear Roundness
Maximum Cytoplasm Radius
Membrane Segmentation Aggressiveness.
   Immunoneutralisation of T14 Antibody with Blocking Peptide

A T14 blocking peptide was stored at −20° C. after receipt. Epitope retrieval was performed using HIER (Heat Induced Epitope Retrieval). The blocking peptide was used at a concentration of 10 ug/mL (anti-T14 Ab concentration of 1 ug/mL). The blocking peptide and anti-T14 pAb was incubated for 1 hour at room temperature.

### Example 1—Detecting T14 in the Skin

The inventors have continued their previous work in this area, and have focused on the toxic peptide, T14, which is derived from the C-terminus of acetylcholinesterase (AChE), which is present as a naturally occurring bioactive molecule in brain tissue.

The inventors have previously shown that T14 in blood and CSF can be used as a biomarker for diagnosing and prognosing a neurodegenerative disorder. Accordingly, the inventors now turned their attention to the skin, to determine whether T14 could be used as a diagnostic and prognostic biomarker in skin for aging and neurodegenerative diseases.

Referring to FIG. 1, there is shown immunohistochemical staining of human Alzheimer's diseased brain tissue at Braak Stages III-IV and VI as a positive control, in order to validate the antibody protocol and ensure T14 peptide detection and staining is clear.

The inventors then detected the levels of T14 positive cells in young (16, 23 and 29 years) and old (53, 54 and 56 years) skin tissue. Surprisingly, the inventors observed that the T14 peptides (SEQ ID No: 3) can be detected in skin. Moreover, the inventors were surprised to observe that the number of T14 positive cells in skin tissue of the old age group was significantly reduced when compared with the young age group, as illustrated in FIGS. 2 and 3. In particular, the average number of T14 positive cells in the young age group was around 172 cells, which decreased to approximately 40 cells in the old age group.

Furthermore, FIG. 4 illustrates the number of T14 positive cells, as well as T14 stain intensity, normalised to the epidermal area. Therefore, these results further demonstrate that in older skin tissue, the total number of T14 positive cells is reduced, but also, the concentration of T14 peptide in these T14 positive cells is reduced.

Additionally, data from the extended skin panel illustrates that T14 expression visually decreases across photo-protected age groups, as shown in FIG. 5A. The quantitative results shown in FIG. 5B also demonstrate that the number of T14 positive cells decrease in aged skin samples compared to young skin samples, even when epidermal area is taken into account.

### Example 2—Diagnosing Neurodegenerative Disease by Detecting T14 in the Skin

The inventors have previously shown that the T14 peptide is a biomarker of cellular development and growth, and that an increase in T14 levels in the brain or CSF of Alzheimer's patients is due to excessive/inappropriate neuronal growth. The inventors have now surprisingly found that T14 can be readily detected in the skin, and that T14 levels are high in young, growing skin, but decreases over time, and drops to lower levels in older skin. Therefore, the inventors postulate that detection of high T14 concentrations in the older skin of a subject means that subject could have Alzheimer's Disease or a predisposition to later developing AD.

As an illustrative example, the following method may be carried out in order to determine whether or not an individual suffers from Alzheimer's disease. Firstly, a skin sample is taken from the patient, for example, by using a punch biopsy, a shave biopsy, an excisional biopsy, or by a non-invasive suction blister method. Briefly, glass chambers are attached to an area of the skin and connected to a vacuum pump, such that epidermis is slowly detached from underlying dermis. An induced blister is then formed that fills with interstitial fluid, which can be extracted with a syringe.

The skin sample may be taken from any area of the individual's body, such as from the arm. The skin sample is then placed in a fixative, such as formaldehyde, and may also be embedded in paraffin to ensure the tissue architecture is maintained. The skin tissue sample is then sectioned, for example into sections of 5 μm in thickness, and then mounted onto a glass slide coated with a tissue adhesive. High heat epitope retrieval is performed and then slides are washed in tris-buffered saline (TBS). The tissue sample is then stained with the anti-T14 antibody, sections are then dehydrated in alcohol washes, run through xylene washes, and cover slipped. The slides are then imaged and the number of T14 positive cells in the skin sample are quantified, and compared with a control patient who does not suffer from Alzheimer's disease. If the number of T14 positive cells in the individual's skin sample is greater than the number of T14 positive cells in the skin sample of the control patient, then this suggests that the individual suffers from, or has a pre-disposition to Alzheimer's disease.

A skin sample may be obtained from the individual at multiple spaced apart time points, for example, every year, every six months, or even every month, and the number of T14 positive cells in the individual's skin sample can be compared with a control patient who does not suffer from Alzheimer's disease at each of these time points.

Advantageously, therefore, an individual can be monitored frequently to determine whether they suffer from Alzheimer's disease, allowing an early diagnosis to be given, and new treatment regime to be introduced.

### Example 3—Quantifying the Age of Skin by Detecting T14 Levels in the Skin

The inventors have surprisingly found that T14 can be readily detected in the skin, and that T14 levels are high in young, growing skin, but decreases over time, and drops to lower levels in older skin. Therefore, the inventors postulate that by analysing the concentration of T14 in the skin, an individual's biological skin age can be quantified and compared with their chronological age. Advantageously, therefore, if the individual's biological skin age is older than their chronological age, they can apply cosmetic treatments, such as moisturisers or anti-aging creams, or change their lifestyle habits, such as by avoiding sunbathing or smoking or excessive alcohol intake, in order to delay or reverse the progression of skin aging.

As an illustrative example, the following method may be carried out in order to quantify the biological age of an individual's skin. Firstly, a skin sample is taken from the patient, for example, by using a punch biopsy, a shave biopsy, an excisional biopsy, or by a non-invasive suction blister method. Briefly, glass chambers are attached to an area of the skin and connected to a vacuum pump, such that epidermis is slowly detached from underlying dermis. An induced blister is then formed that fills with interstitial fluid, which can be extracted with a syringe.

The skin sample may be taken from any area of the individual's body, such as from the arm. The skin sample is then placed in a fixative, such as formaldehyde, and may also be embedded in paraffin to ensure the tissue architecture is maintained. The skin tissue sample is then sectioned, for example into sections of 5 μm in thickness, and then mounted onto a glass slide coated with a tissue adhesive. High heat epitope retrieval is performed and then slides are washed in tris-buffered saline (TBS). The tissue sample is then stained with the anti-T14 antibody, sections are then dehydrated in alcohol washes, run through xylene washes, and cover slipped. The slides are then imaged and the number of T14 positive cells in the test skin sample are quantified, and compared with a skin sample obtained from the subject at an earlier time point, i.e. a younger reference skin sample. The younger reference skin sample may be obtained from the subject one month, six months, or even a year before the test sample is obtained. If the number of T14 positive cells in the individual's test skin sample is lower than the number of T14 positive cells in the younger reference skin sample, then this suggests that the individual's biological skin age is older, and therefore, their skin has suffered from aging.

For example, if a test skin sample is obtained from an individual a year after the reference skin sample was taken, then chronologically, the skin has aged by one year.

However, if the individual has been excessively sunbathing or drinking alcohol, then this will have accelerated the skin aging process, and as such, the biological age of the skin will have increased by more than a year, for example by two years. This biological aging of the skin would be detected by a decrease in the number of T14 positive cells in the skin test sample compared to the reference skin sample.

A skin sample may be obtained from the individual at multiple spaced apart time points, for example, every year, every six months, or even every month, and the number of T14 positive cells in the individual's skin sample can be compared with the younger reference skin sample at each of these time points.

Advantageously, therefore, an individual can frequently monitor the biological aging of their skin to determine whether they should apply cosmetic treatments, such as moisturisers or anti-aging creams, or change their lifestyle habits, such as by avoiding sunbathing or smoking or excessive alcohol intake, to delay or reverse the progression of skin aging.

The subject can then take positive step to delay their skin from aging any further and also to reverse the aging, for example by applying moisturiser, or sunblock and from changing certain lifestyle habits, such as avoiding the sun.

### Example 4—T14 Expression in Aged Photo-Protected Vs Aged Photo-Exposed Skin

The inventors also set out to demonstrate that T14 could be regarded as an index reflecting not just age but also photo-induced aging. As illustrated in FIG. 6a, T14 expression is decreased in photo-exposed skin compared to photo-protected skin.

Additionally, image analysis confirmed that photo-exposed skin has a significant reduction in the number of T14 positive epidermal cells even when epidermal area is taken into account (FIG. 6B). Accordingly, these data illustrate that there is a significant difference, within the same age range, of photo-protected versus photo-exposed skin, demonstrating that T14 declines as an index not only of age (i.e. chronological/calendar age), but also of aging. Therefore, this observation suggests that T14 is actively involved in the actual rate of cell renew, and/or in cellular growth mechanisms rather than merely reflecting calendar age.

### Example 5—Localisation of T14 in the Epidermis

The inventors next set out to determine the localisation of T14 staining in the epidermis. As illustrated in FIG. 7, T14 is mainly localised in the nucleus in young skin samples, however, it is also present in the cytoplasm in aged skin samples. Additionally, FIG. 8 shows that the number of T14 positive nuclei significantly decreases with age in both photo-protected and photo-exposed groups. The inventors also observed that the number of cells containing T14 within the cytoplasm significantly increases with age in photo-protected groups. However, the number of cells containing T14 within the cytoplasm significantly decreases in aged photo-exposed samples compared to aged photo-protected samples.

### Example 6—Immunoneutralisation of T14 Antibody to Confirm T14 Staining in the Epidermis

Finally, the inventors set out to block binding of the anti-T14 polyclonal antibody to the T14 epitope, using a blocking peptide, to confirm T14 staining in the epidermis. As illustrated in FIG. 8, the peptide successfully blocked binding of the anti-T14 polyclonal antibody to the T14 epitope, for both young and aged skin samples.

### CONCLUSIONS

The inventors have previously demonstrated that T14 levels increase in Alzheimer's patients, and in particular, that T14 in CSF or blood may be used as a robust diagnostic biomarker for early detection of the disease. However, accessing nervous tissue for the purposes of biomarker acquisition presents numerous difficulties, and the analysis of biomarkers in samples such as CSF or blood, requires more complex and invasive sample collection from the patient.

The inventors have now surprisingly demonstrated that the T14 peptide (SEQ ID No: 3) can be detected in skin per se, and even more surprisingly, that T14 expression significantly decreases with age. This was unexpected. As such, these data clearly demonstrate that the T14 peptide can be used as a robust biomarker in skin, for detecting skin aging, and for identifying individuals who suffer from neurodegenerative disease, such as Alzheimer's disease or Parkinson's disease. Advantageously, therefore, the claimed invention provides an easier, quicker and less invasive method of diagnosing neurodegenerative disorders, or detecting skin aging, compared to methods used currently which require more invasive sampling of the CSF or blood.

1. (canceled)

2. A method for treating a subject who suffers from a neurodegenerative disorder, or a pre-disposition thereto, the method comprising:
(i) analysing, in a skin sample obtained from the subject, a concentration of a peptide of SEQ ID No: 3, or a variant or fragment thereof, and comparing this concentration with a reference for the concentration of a peptide of SEQ ID No: 3, or a variant or fragment thereof in a subject who does not suffer from a neurodegenerative disease, wherein an increase in the concentration of the peptide of SEQ ID No: 3, or a variant or fragment thereof, indicates that the subject suffers from a neurodegenerative disorder, or has a pre-disposition thereto; and
(ii) administering to the subject a therapeutic agent or putting the subject on a specialised diet when an increase in the concentration of SEQ ID No: 3 is detected in step (i), wherein the therapeutic agent or the specialised diet prevents, reduces or delays progression of a neurodegenerative disorder.

3. A method for detecting SEQ ID No: 3, or a variant or fragment thereof in a test subject, the method comprising detecting the concentration of the SEQ ID No: 3, or a variant or fragment thereof in a skin sample from the test subject.

4. A kit for diagnosing a test subject suffering from a neurodegenerative disorder, or a pre-disposition thereto, or for providing a prognosis of the test subject's condition, the kit comprising detection means for determining the concentration of a peptide of SEQ ID No: 3, or a variant or fragment thereof, present in a skin sample from the test subject, and a reference for the concentration of a peptide of SEQ ID No: 3, or a variant or fragment thereof in a subject who does not suffer from a neurodegenerative disease, wherein an increase in the concentration of the peptide of SEQ ID No: 3, or a variant or fragment thereof, in the skin sample indicates that the test subject suffers from a neurodegenerative disorder, or a pre-disposition thereto.

5. The method according to claim 2, wherein the neurodegenerative disorder is selected from a group consisting of Alzheimer's disease; Parkinson's disease; Huntington's disease; Motor Neurone disease; Spinocerebellar type 1, type 2, and type 3; Amyotrophic Lateral Sclerosis (ALS); and Frontotemporal Dementia.

6. The method according to claim 2, wherein the neurodegenerative disorder is Alzheimer's disease.

7. (canceled)

8. A method for detecting skin aging in a subject, the method comprising analysing, in a skin sample obtained from the subject, the concentration of a peptide of SEQ ID No: 3, or a variant or fragment thereof, and comparing this concentration with a reference for the concentration of a peptide of SEQ ID No: 3, or a variant or fragment thereof in a younger skin sample wherein a decrease in the concentration of the peptide of SEQ ID No: 3, or a variant or fragment thereof, indicates aging of the subject's skin.

9. The method according to claim 8, wherein the method comprises quantifying the age of a subject's skin.

10. The method according to claim 8, wherein the method comprises obtaining a skin sample from the subject every ten, eleven or 12 months and comparing the concentration of a peptide of SEQ ID No: 3, or a variant or fragment thereof, with the concentration of a peptide of SEQ ID No: 3, or a variant or fragment thereof in the younger skin sample.

11. A method according to claim 8, wherein the method comprises obtaining a skin sample from the subject every month or week and comparing the concentration of a peptide of SEQ ID No: 3, or a variant or fragment thereof, with the concentration of a peptide of SEQ ID No: 3, or a variant or fragment thereof in the younger skin sample.

12. A kit for detecting skin aging in a subject, the kit comprising detection means for determining the concentration of a peptide of SEQ ID No: 3, or a variant or fragment thereof, present in a skin sample from the [test] subject, and a reference for the concentration of a peptide of SEQ ID No: 3, or a variant or fragment thereof in a younger skin sample, wherein a decrease in the concentration of the peptide of SEQ ID No: 3, or a variant or fragment thereof, in the skin sample indicates aging of the subject's skin.

13. The kit according to claim 12, wherein the kit comprises means for quantifying the age of a subject's skin.

14. The method according to claim 8, wherein the reference for the concentration of a peptide of SEQ ID No: 3, or a variant or fragment thereof in a younger skin sample is taken from the same subject as the skin sample but at an earlier time point.

15. The method according to claim 14, wherein the younger skin sample is taken from the subject at least one month before the skin sample, such that a direct comparison of the concentration of the peptide of SEQ ID No: 3, or a variant or fragment thereof can be made between the skin sample and the younger reference skin sample.

16. The method according to claim 2, wherein a detection means for analysing the concentration of a peptide of SEQ ID No: 3, or a variant or fragment thereof, comprises an antibody or antigen binding fragment thereof, or wherein the detection means comprises the use of immunohistochemistry.

17. (canceled)

18. The method according to claim 2, wherein the skin sample comprises skin epidermis and/or skin dermis, optionally wherein the skin sample comprises stratum basale, stratum spinosum, stratum granulosum, stratum lucidum and/or stratum corneum.

19. The method according to claim 2, wherein the skin sample comprises at least one keratinocyte.

20. The method according to claim 2, wherein the skin sample is obtained using a skin punch biopsy, using a suction blister method, from scraped cells, from skin shave biopsies, by skin stripping, and/or through fluid collected under pressure using reverse iontophoresis.

21. The kit according to claim 4, wherein the kit comprises means for obtaining the skin sample, selected from at least one of a razor, a scalpel, a blade, a biopsy punch, adhesive tape, a glass chamber, a vacuum pump and/or a syringe.

22. The method according to claim 8, wherein the method comprises analysing the concentration of a peptide of SEQ ID No: 3, or a variant or fragment thereof, in the nuclei of epidermal cells in the skin sample, wherein a decrease in the concentration of the peptide of SEQ ID No: 3, or a variant or fragment thereof in the nuclei of epidermal cells in the skin sample compared to the younger skin sample, indicates aging of the subject's skin, wherein the aging is biological and/or chronological aging.

23. The method according to claim 8, wherein the method comprises analysing the concentration of a peptide of SEQ ID No: 3, or a variant or fragment thereof, in the cytoplasm of epidermal cells in the skin sample, wherein:
(i) an increase in the concentration of the peptide of SEQ ID No: 3, or a variant or fragment thereof in the cytoplasm of epidermal cells in the skin sample compared to the younger skin sample, indicates aging of the subject's skin, wherein the aging is chronological aging; or
(ii) a decrease in the concentration of the peptide of SEQ ID No: 3, or a variant or fragment thereof in the cytoplasm of epidermal cells in the skin sample compared to the younger skin sample, indicates aging of the subject's skin, wherein the aging is biological aging.
